(19) United States
(12) Patent Application Publication
(10) Pub. No.: US 2014/0081098 A1
(43) Pub. Date: Mar. 20, 2014

(54) SENSOR SYSTEM

(71) Applicants: Kristi Cohrs, Englewood, CO (US); James Nicholas Watson, Dunfermline (GB); Paul Stanley Addison, Edinburgh (GB); Mark Su, Boulder, CO (US)

(72) Inventors: Kristi Cohrs, Englewood, CO (US); James Nicholas Watson, Dunfermline (GB); Paul Stanley Addison, Edinburgh (GB); Mark Su, Boulder, CO (US)

(73) Assignee: Nellcor Puritan Bennett LLC, Boulder, CO (US)

(21) Appl. No.: 13/618,227

(22) Filed: Sep. 14, 2012

(51) Int. Cl. A61B 5/024

(52) U.S. Cl. CPC A61B 5/02444
USPC 600/324; 600/500

(57) ABSTRACT

A sensor system is provided for determining a pulse transit time measurement of a patient. The sensor system includes a carotid sensor device configured to be positioned on a neck of the patient over a carotid artery of the patient. The carotid sensor device is configured to detect a plethysmograph waveform from the carotid artery. The sensor system includes a temporal sensor device that is operatively connected to the carotid sensor device. The temporal sensor device is configured to be positioned on the patient over a temporal artery of the patient. The temporal sensor device is configured to detect a plethysmograph waveform from the temporal artery.

### FIELD

Embodiments of the present disclosure generally relate to medical devices, and more particularly to the use of sensor systems for monitoring various physiological characteristics of a patient.

### BACKGROUND

Various methods are used to determine the cardiac output of a patient. For example, a pulse transit time of a patient may be used, along with other physiological variables, to determine cardiac output of a patient. Pulse transit time is typically measured using an electrocardiogram (ECG) and a pulse oximeter sensor on a finger or other digit of the patient. Moreover, there may be a variable delay between the electrical pulses detected by the ECG and the mechanical ejection of blood from the patient's heart. Moreover, the pulse wave being measured travels through a relatively complicated and long vascular path due to the relatively large distance between the ECG measurements taken at the patient's heart and the pulse oximetry measurements taken at the patient's digit, for example. The variable delay between the ECG and the mechanical activity of the heart and/or the relatively long and complicated vascular path of the pulse wave may cause errors in the measured pulse transit time, which may lead to erroneous predictions regarding cardiac output, which may, in turn, lead to false diagnoses, for example.

### SUMMARY

Certain embodiments provide a sensor system for determining a pulse transit time measurement of a patient. The sensor system includes a carotid sensor device configured to be positioned on a neck of the patient over a carotid artery of the patient. The carotid sensor device is configured to detect a plethysmograph waveform from the carotid artery. The sensor system includes a temporal sensor device that is operatively connected to the carotid sensor device. The temporal sensor device is configured to be positioned on the patient over a temporal artery of the patient. The temporal sensor device is configured to detect a plethysmograph waveform from the temporal artery.

The sensor system may include a pulse transit time determination module that is operatively connected to the carotid sensor device and to the temporal sensor device. The pulse transit time determination module may be configured to determine the pulse transit time measurement based, at least in part, on the plethysmograph waveforms from the carotid and temporal arteries.

The temporal sensor device may include a housing and a sensor held by the housing. The sensor may be configured to detect the plethysmograph waveform from the temporal artery. The housing may define an ear clip that is configured to be received around the base of an ear of the patient. The ear clip may have an end that is configured to be positioned over the temporal artery of the patient. The ear clip may extend outward from the end along a path that is configured to wrap around a top of a base of an ear of the patient. The ear clip may include a lower extension that is configured to wrap around a back of the base of the patient's ear. The ear clip may be resiliently compressible around the base of the patient's ear.

The carotid sensor device may include a housing and a sensor held by the housing. The sensor may be configured to detect the plethysmograph waveform from the carotid artery. The housing may include a surface that includes a shape that is complementary with a shape of the patient's neck. The housing may include a surface that includes a convex segment that is configured to engage skin of the patient's neck over the carotid artery.

The sensor system may include a cable. The carotid sensor device may be operatively connected to the temporal sensor device via the cable.

The sensor system may include a pulse-oximeter sensor device that is held by the temporal sensor device such that the pulse-oximeter sensor device is configured to be positioned on a lobe of an ear of the patient for measuring pulse oximeter waveforms.

The carotid sensor device and/or the temporal sensor device may include an adhesive for affixing the device to skin of the patient.

The carotid sensor device may include a photoplethysmograph (PPG) sensor, a blood pressure sensor, a pressure transducer, an optical PPG sensor, a photoacoustic sensor, and/or a photon density wave sensor.

The temporal sensor device may include a photoplethysmograph (PPG) sensor, a blood pressure sensor, a pressure transducer, an optical PPG sensor, a photoacoustic sensor, and/or a photon density wave sensor.

The carotid sensor device and/or the temporal sensor device may be a non-invasive sensor device and/or a disposable, single use, sensor device.

Certain embodiments provide a method for determining a pulse transit time of a patient using a sensor system. The method may include affixing a carotid sensor device to a neck of the patient over a carotid artery of the patient, and affixing a temporal sensor device to the patient over a temporal artery of the patient. The method may include detecting a plethysmograph waveform from the carotid artery of the patient using the carotid sensor device, and detecting a plethysmograph waveform from the temporal artery of the patient using the temporal sensor device. The method may include determining the pulse transit time measurement based, at least in part, on the plethysmograph waveforms from the carotid and temporal arteries.

Certain embodiments provide a temporal sensor device that may include a housing having an internal compartment and a temporal segment. The housing may include an ear clip that is configured to wrap around the base of an ear of a patient such that the temporal segment of the housing is positioned over a temporal artery of the patient. A sensor may be held within the internal compartment of the housing at the temporal segment of the housing such that the sensor is configured to detect a plethysmograph waveform from the temporal artery when the ear clip is wrapped around the base of the patient's ear.

Certain embodiments of the present disclosure may provide a sensor system that is more accurate and reliable than previous systems for determining pulse transit time measurements, cardiac output, stroke volume, vascular resistance, and/or the like. Embodiments of the present disclosure may provide a sensor system for determining pulse transit time measurements that includes at least two sensors that are spaced apart along the vasculature of the patient. Certain embodiments of the present disclosure may provide a sensor system that detects plethysmograph waveforms at relatively close locations having a path therebetween that is relatively direct and uncomplicated. Certain embodiments of the present disclosure may provide a sensor system that is less susceptible to vasoconstriction. Certain embodiments of the present disclosure may provide a sensor system that enables sensors to detect plethysmograph waveforms from carotid and temporal arteries of a patient in a relatively non-invasive manner.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

FIG. 1 illustrates a simplified block diagram of an exemplary embodiment of a sensor system for determining a pulse transit time of a patient.

FIG. 2 illustrates a more detailed block diagram of an exemplary embodiment of the sensor system shown in FIG. 1.

FIG. 3 illustrates an exemplary plethysmograph waveform obtained using a photoplethysmograph (PPG) sensor of the sensor system shown in FIGS. 1 and 2.

FIG. 4 illustrates an exemplary plethysmograph waveform obtained using a blood pressure sensor of the sensor system shown in FIGS. 1 and 2.

FIG. 5 is an elevational view of an exemplary embodiment of a temporal sensor device of the sensor system shown in FIGS. 1 and 2.

FIG. 6 is a perspective view of an exemplary embodiment of a carotid sensor device of the sensor system shown in FIGS. 1 and 2.

FIG. 7 is an elevational view illustrating the sensor system shown in FIGS. 1 and 2 operatively connected to a patient.

FIG. 8 is a graph illustrating an exemplary ensemble pressure pulse.

FIG. 9 is an elevational view of another embodiment of a sensor system.

FIG. 10 is a flowchart illustrating an exemplary embodiment of a method for determining a pulse transit time measurement of a patient.

### DETAILED DESCRIPTION

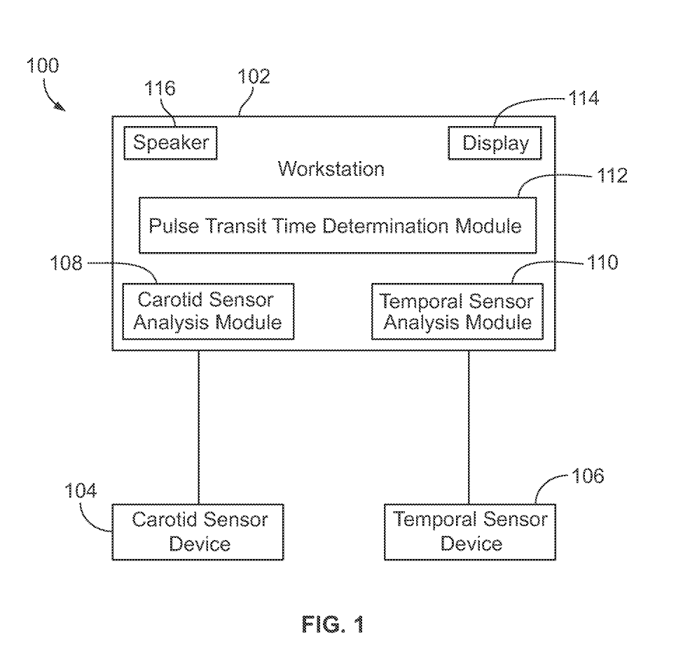

FIG. 1 illustrates a simplified block diagram of an exemplary embodiment of a sensor system 100 for determining a pulse transit time measurement of a patient. The pulse transit time measurement may be used to determine various physiological parameters of the patient, such as, but not limited to, cardiac output, stroke volume, vascular resistance, and/or the like. A pulse transit time may be inversely proportional to a pulse wave velocity when measured over fixed path lengths. Therefore, methods employing the pulse transit time may, in some alternative embodiments, be implemented using pulse wave velocity measurements.

The system 100 may include a workstation 102 operatively connected to a carotid sensor device 104 and a temporal sensor device 106. As will be described below, the carotid sensor device 104 is configured to be positioned on a neck of the patient over a carotid artery of the patient for detecting a plethysmograph waveform from the carotid artery, while the temporal sensor device 106 is configured to be positioned on the patient over a temporal artery of the patient for detecting a plethysmograph waveform from the temporal artery. The workstation 102 may be operatively connected to each of the carotid sensor device 104 and the temporal sensor device 106 through cables, wireless connections, and/or the like.

The workstation 102 may be or otherwise include one or more computing devices, such as standard computer hardware. The workstation 102 may include one or more modules and control units, such as processing devices that may include one or more microprocessors, microcontrollers, integrated circuits, memory, such as read-only and/or random access memory, and the like. For example, the workstation 102 may include a carotid sensor analysis module 108, a temporal sensor analysis module 110, and/or a pulse transit time determination module 112. The carotid sensor analysis module 108 may be configured to analyze a plethysmograph waveform received from the carotid sensor device 104. The temporal sensor analysis module 110 may be configured to analyze a plethysmograph waveform received from the temporal sensor device 106. The pulse transit time determination module 112 may be configured to determine pulse transit time based on signals analyzed by the carotid sensor analysis module 108 and the temporal sensor analysis module 110.

While shown as separate and distinct modules, the carotid sensor analysis module 108, the temporal sensor analysis module 110, and the pulse transit time determination module 112 may alternatively be integrated into a single module, processor, controller, integrated circuit, and/or the like. For example, the pulse transit time determination module 112 may include the carotid sensor analysis module 108 and the temporal sensor analysis module 110. Additionally, the carotid sensor analysis module 108 may be part of the carotid sensor device 104, while the temporal sensor analysis module 110 may be part of the temporal sensor device 106, instead of being separately and distinctly part of the workstation 102. In such an embodiment, fully-analyzed plethysmograph waveforms may be sent to the pulse transit time determination module 112 from the carotid sensor device 104 and the temporal sensor device 106.

Although shown as being a component of the workstation 102, the pulse transit time determination module 112 may alternatively be a monitor that is separate and distinct from the workstation 102. In embodiments wherein the pulse transit time determination module 112 is separate and distinct from the workstation 102, the module 112 may be communicatively coupled to the workstation 102 via a cable (not shown) and/or may communicate wirelessly with the workstation 102. Additionally, the module 112 and/or the workstation 102 may be coupled to a network to enable the sharing of information with servers, other workstations, and/or the like.

The workstation 102 may also include a display 114, such as, but not limited to, a cathode ray tube display, a flat panel display, a liquid crystal display (LCD), a light-emitting diode (LED) display, a plasma display, and/or any other type of monitor. The workstation 102 may be configured to calculate physiological parameters and to show information from the carotid sensor device 104, the temporal sensor device 106, and/or from other medical monitoring devices or systems (e.g., the pulse-oximeter sensor device 402 shown in FIG. 9) on the display 114. For example, the workstation 102 may be configured to display blood pressure of the patient generated from the carotid sensor device 104 and/or the temporal sensor device 106, plethysmograph waveforms generated from the carotid sensor device 104 and/or the temporal sensor device 106, cardiac output of the patient, stroke volume, vascular resistance, and/or the like on the display 114. The workstation 102 may include a speaker 116 configured to provide an audible sound that may be used in various embodiments, such as, but not limited to, sounding an audible alarm in the event that one or more physiological parameters are outside a predefined normal range.

The workstation 102 may include any suitable computer-readable media used for data storage. Computer-readable media are configured to store information that may be interpreted by the workstation 102 in general, and by the pulse transit time determination module 112, the carotid sensor analysis module 108, and the temporal sensor analysis module 110, in particular. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

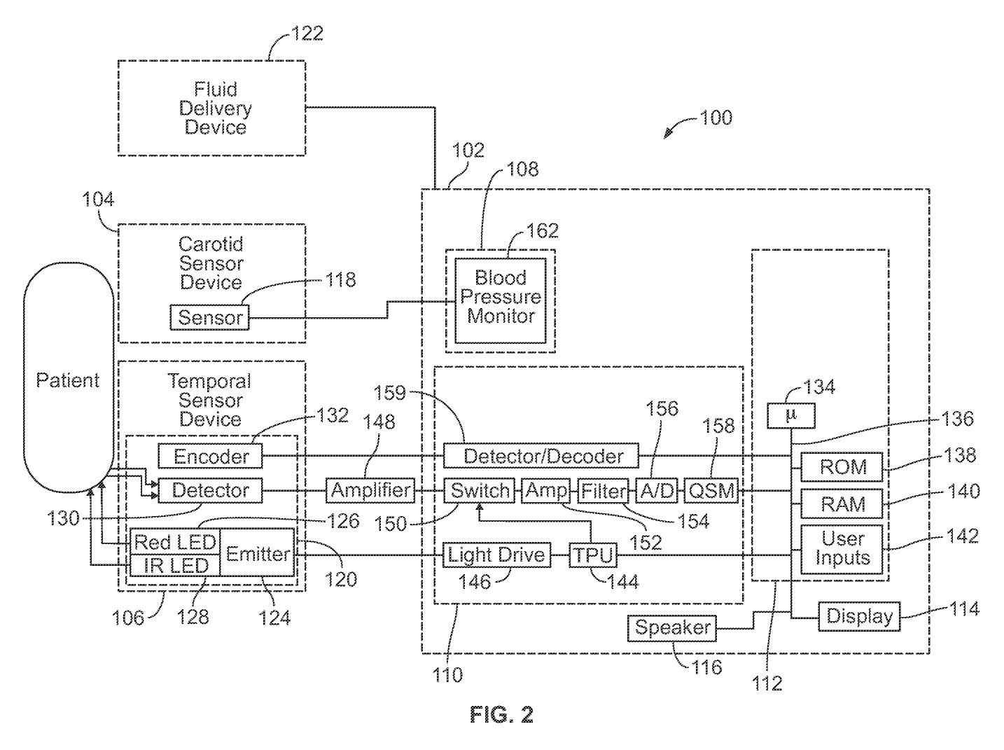

FIG. 2 illustrates a more detailed block diagram of an exemplary embodiment of the sensor system 100. The carotid sensor device 104 and the temporal sensor device 106 include respective sensors 118 and 120. As will be described below, the sensor 118 of the carotid sensor device 104 is configured to detect a plethysmograph waveform from a carotid artery of the patient, while the sensor 120 of the temporal sensor device 106 is configured to detect a plethysmograph waveform from a temporal artery of the patient.

Each sensor 118 and 120 may be any type(s) of sensor that is configured to detect a plethysmograph waveform from the corresponding artery. For example, in some embodiments the sensor 118 of the carotid sensor device 104 is a blood pressure sensor, while in other embodiments the sensor 118 is a photoplethysmograph (PPG) sensor. Moreover, and for example, in some embodiments the sensor 120 of the temporal sensor device 106 is a blood pressure sensor, while in other embodiments the sensor 120 is a PPG sensor. In still other embodiments, the sensor 118 and/or the sensor 120 includes both a blood pressure sensor and a PPG sensor. Examples of suitable blood pressure sensors include, but are not limited to, pressure transducers, piezoelectric transducers, and/or the like. Examples of suitable PPG sensors include, but are not limited to, optical PPG sensors, photoacoustic (PA) sensors, photon density wave sensors, and/or the like. Each of the sensors 118 and 120 may include a plurality of sensors forming a sensor array in place of a singe sensor.

The carotid sensor device 104 and/or the temporal sensor device 106 may be operatively connected to the pulse transmit time determination module 112 for drawing power from the module 112. In addition or alternatively, the devices 104 and/or 106 may include a battery and/or similar power supply (not shown).

The sensor system 100 may include a fluid delivery device 122 that is configured to deliver fluid to the patient. The fluid delivery device 122 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 122 may be configured to adjust the quantity or concentration of fluid delivered to a patient. The fluid delivery device 122 may be communicatively coupled to the workstation 102 and/or the pulse transit time determination module 112 via a cable (not shown), wirelessly, and/or the like. In some embodiments, the sensor system 100 includes a skin temperature measuring device (not shown) for measuring the temperature of the patient's skin at one or more various locations.

In the exemplary embodiment of FIG. 2, the sensor 118 of the carotid sensor device 104 is a blood pressure sensor, while the sensor 120 of the temporal sensor device 106 is PPG sensor. An exemplary embodiment of the system 100 wherein the sensor 120 is a PPG sensor will now be described. It should be understood that the discussion of the sensor 120 as a PPG sensor is applicable to embodiments wherein the sensor 118 is a PPG sensor. The sensor 120 includes an emitter 124 that is configured to emit light into tissue and/or blood of the patient. For example, the emitter 124 may be configured to emit light at two or more wavelengths (e.g., red and infrared) into the tissue and/or blood of the patient. The emitter 124 may include a red light-emitting light source such as a red light-emitting diode (LED) 126 and an infrared light-emitting light source such as an infrared LED 128 for emitting light into the tissue and/or blood at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

In other embodiments, the sensor 120 may be configured to emit more or less than two wavelengths of light into the tissue and/or blood of the patient. Further, the sensor 120 may be configured to emit wavelengths of light other than red and infrared into the tissue and/or blood of the patient. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the sensor 120.

The sensor 120 also includes a detector 130 that is configured to detect emitted light from the emitter 124 that emanates from the tissue and/or blood after passing therethrough. The detector 130 may be configured to be specifically sensitive to the chosen targeted energy spectrum of the emitter 124. The detector 130 may be configured to detect the intensity of light at the red and infrared wavelengths. Alternatively, each sensor in an array may be configured to detect an intensity of a single wavelength. In operation, light may enter the detector 130 after passing through the patient's blood and/or tissue. The detector 130 may convert the intensity of the received light into an electrical signal. The light intensity may be directly related to the absorbance and/or reflectance of light in the tissue 130. For example, when more light at a certain wavelength is absorbed or reflected, less light of that wavelength is received from the tissue by the detector 130. After converting the received light to an electrical signal, the detector 130 may send the signal to the temporal sensor analysis module 110 and/or the pulse transit time determination module 112 for calculation of physiological parameters based on the absorption of the red and infrared wavelengths in the tissue and/or blood.

In an embodiment, an encoder 132 may store information about the sensor 120, such as sensor type (for example, whether the sensor is intended for placement on a neck or head of the patient) and the wavelengths of light emitted by the emitter 124. The stored information may be used by the temporal sensor analysis module 110 and/or the pulse transit time determination module 112 to select appropriate algorithms, lookup tables and/or calibration coefficients for calculating physiological parameters of the patient. The encoder 132 may store or otherwise contain information specific to a patient, such as, for example, the patient's age, weight, diagnosis, and/or the like. The information may allow the modules 110 and/or 112 to determine, for example, patient-specific threshold ranges related to the patient's physiological parameter measurements, and to enable or disable additional physiological parameter algorithms. The encoder 132 may, for example, be a coded resistor that stores values corresponding to the type of sensor 120 or the types of each sensor in the sensor array, the wavelengths of light emitted by emitter 124 on each sensor of the sensor array, and/or the patient's characteristics. Optionally, the encoder 132 may include a memory in which one or more of the following may be stored for communication to the modules 110 and/or 112: the type of the sensor 120, the wavelengths of light emitted by emitter 124, the particular wavelength each sensor in the sensor array is monitoring, a signal threshold for each sensor in the sensor array, any other suitable information, or any combination thereof.

Signals from the detector 130 and the encoder 132 may be transmitted to the temporal sensor analysis module 110 and/or the pulse transit time determination module 112. The modules 110 and/or 112 may include a general-purpose control unit, such as a microprocessor 134 connected to an internal bus 136. The microprocessor 134 may be configured to execute software, which may include an operating system and one or more applications, as part of performing the functions described herein. A read-only memory (ROM) 138, a random access memory (RAM) 140, user inputs 142, the display 114, and/or the speaker 116 may also be operatively connected to the bus 136.

The RAM 140 and the ROM 138 are illustrated by way of example, and not limitation. Any suitable computer-readable media may be used in the system for data storage. Computer-readable media are configured to store information that may be interpreted by the microprocessor 134. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The temporal sensor analysis module 110 and/or the pulse transit time determination module 112 may include a time processing unit (TPU) 144 configured to provide timing control signals to a light drive circuitry 146, which may control when the emitter 124 is illuminated and multiplexed timing for the red LED 126 and the infrared LED 128. The TPU 144 may also control the gating-in of signals from the detector 130 through an amplifier 148 and a switching circuit 150. The signals are sampled at the proper time, depending upon which light source is illuminated. The received signal from the detector 130 may be passed through an amplifier 152, a low pass filter 154, and an analog-to-digital converter 156. The digital data may then be stored in a queued serial module (QSM) 158 (or buffer) for later downloading to RAM 140 as QSM 158 fills up. In an embodiment, there may be multiple separate parallel paths having amplifier 152, filter 154, and A/D converter 156 for multiple light wavelengths or spectra received.

The microprocessor 134 may be configured to determine the patient's physiological parameters, such as, but not limited to, pulse transit time, cardiac output, stroke volume, vascular resistance, and/or the like, using various algorithms and/or look-up tables based on the value(s) of the received signals and/or data corresponding to the light received by the detector 130. The signals corresponding to information about a patient, and regarding the intensity of light emanating from the patient's tissue and/or blood over time, may be transmitted from the encoder 132 to a decoder 159. The transmitted signals may include, for example, encoded information relating to patient characteristics. The decoder 159 may translate the signals to enable the microprocessor 134 to determine the thresholds based on algorithms or look-up tables stored in the ROM 138. The user inputs 142 may be used to enter information about the patient, such as, but not limited to, age, weight, height, diagnosis, medications, treatments, and/or the like. The display 114 may show a list of values that may generally apply to the patient, such as, but not limited to, age ranges or medication families, which the user may select using the user inputs 142.

PPG sensors that may be suitable for use as the sensor 118 and/or the sensor 120 are further described in United States Patent Application Publication No. 2012/0053433, entitled “System and Method to Determine SpO₂ Variability and Additional Physiological Parameters to Detect Patient Status,” United States Patent Application Publication No. 2012/0029320, entitled “Systems and Methods for Processing Multiple Physiological Signals,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

Although shown as being a component of the sensor 120, the encoder 132 may alternatively be a component of the temporal sensor analysis module 110 or of the pulse transit time determination module 112. While shown as being components of the temporal sensor analysis module 110, the decoder 159, the switching circuit 150, the light drive circuitry 146, the amplifier 148, the amplifier 152, the low pass filter 154, the converter 156, the TPU 144, and the QSM 158 may each be a component of the temporal sensor device 106 or the pulse transit time determination module 112. Moreover, although shown as being components of the pulse transit time determination module 112, the microprocessor 134, the bus 136, the ROM 138, the RAM 140, and the user inputs 142 may each be a component of the sensor 120 or the temporal sensor device 106.

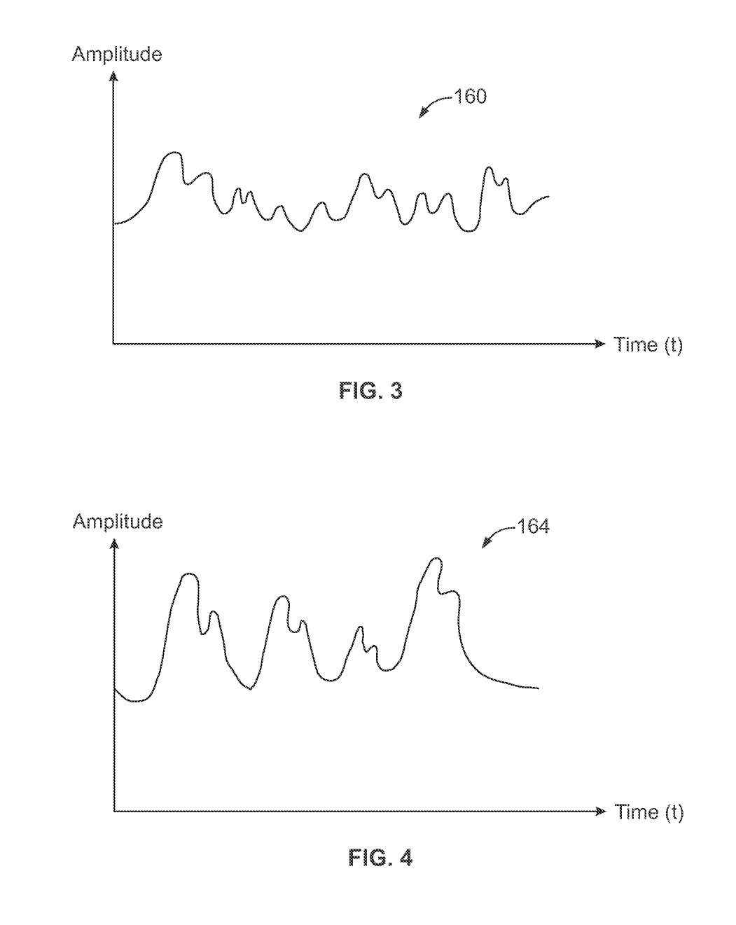

FIG. 3 illustrates an exemplary plethysmograph waveform 160 obtained using a PPG sensor as the sensor 120 (shown in FIGS. 2, 5, and 7). Specifically, the plethysmograph waveform 160 is generated by the sensor 120 of the temporal sensor device 106 (shown in FIGS. 1, 2, 5, and 7). The plethysmograph waveform 160 may be analyzed by the temporal sensor device 106, the temporal sensor analysis module 110 (shown in FIGS. 1 and 2), and/or the pulse transmit time determination module 112 (shown in FIGS. 1 and 2). For example, the microprocessor 134 may analyze the plethysmograph waveform 160 generated by the sensor 120. The plethysmograph waveform 160 may be displayed on the display 114 (shown in FIGS. 1 and 2).

Referring again to FIG. 2, as described above, the sensor 118 of the carotid sensor device 104 is a blood pressure sensor in the exemplary embodiment of FIG. 2. An exemplary embodiment of the system 100 wherein the sensor 118 is a blood pressure sensor will now be described. It should be understood that the discussion of the sensor 118 as a blood pressure sensor is applicable to embodiments wherein the sensor 120 is a blood pressure sensor. The sensor 118 is configured to detect the pressure exerted by circulating blood within vasculature of the patient. Specifically, the sensor 118 is configured to detect pressure pulses of blood within the carotid artery of the patient. The sensor 118 may be configured to detect blood pressure in real time, rather than through intermittent measurement. In some embodiments, the sensor 118 may be configured to detect the effect of blood pressure in real time. For example, the effect of blood pressure in real time may be the mechanical motion of the sensor site (i.e., the location along the patient's skin where the sensor 118 is affixed) as the pressure pulses transit the sensor site. In another example, the effect may be the increase in volume of blood under the sensor 118 as the pressure pulses transit the sensor site.

The carotid sensor analysis module 108 is operatively connected to the sensor 118. The carotid sensor analysis module 108 may be communicatively coupled to the sensor 118 via a cable, wirelessly, and/or the like. The carotid sensor analysis module 108 may be in communication with the pulse transit time determination module 112. The carotid sensor analysis module 108 may be communicatively coupled to the workstation 102 and/or the pulse transit time determination module 112 via a cable, wirelessly, and/or the like. Additionally, the carotid sensor analysis module 108 may be coupled to a network to enable the sharing of information with servers, other workstations, and/or the like.

The carotid sensor analysis module 108 may be configured to determine a blood pressure signal of the patient at the carotid artery based at least in part on data received from the sensor 118. For example, the blood pressure signal determined by the carotid sensor analysis module 108 may be in the form of a plethysmograph waveform detected by the sensor 118. The blood pressure signal determined by the carotid sensor analysis module 108 may be or include a motion signal. The blood pressure signal determined by the carotid sensor analysis module 108 may be or include a signal indicative of mean arterial pressure (MAP), pulse pressure (PP), diastolic pressure, diastolic pressure variations, systolic pressure, systolic pressure variations, and/or the like. Waveforms detected by the sensor 118 and/or blood pressure signals determined using the sensor 118 and the carotid sensor analysis module 108 may be displayed on the display 114.

In some embodiments, the data received from the sensor 118 is passed through a switching circuit 161, an amplifier 162, a low pass filter 163, and/or an analog-to-digital converter 165. The digital data may then be stored in a QSM 167 (or buffer) for later downloading to RAM 140 as QSM 167 fills up. In addition or alternatively to the components 161, 162, 163, 165, and/or 167, the carotid sensor analysis module 108 may include one or more other modules and/or control units, such as, but not limited to, processing devices that may include one or more microprocessors, microcontrollers, integrated circuits, memory (e.g., read-only and/or random access memory), and/or the like.

Although shown as being a component of the carotid sensor analysis module 108, each of the components 161, 162, 163, 165, and/or 167 may alternatively be a component of the carotid sensor device 104, the temporal sensor analysis module 110, and/or the pulse transit time determination module 112. For example, in some alternative embodiments, the modules 108 and 110 share a switching circuit, an amplifier, a low pass filter, an analog-to-digital converter, and/or a QSM.

FIG. 4 illustrates an exemplary plethysmograph waveform 164 obtained using a blood pressure sensor as the sensor 118 (shown in FIGS. 2, 6, and 7). Specifically, the plethysmograph waveform 164 represents the blood pressure measurement generated by the sensor 118 and the blood pressure monitor 162. The plethysmograph waveform 164 may be analyzed by the carotid sensor device 104 (shown in FIGS. 1, 2, 6, and 7), the carotid sensor analysis module 108 (shown in FIGS. 1 and 2), and/or the pulse transmit time determination module 112 (shown in FIGS. 1 and 2). For example, the microprocessor 134 may analyze the plethysmograph waveform 164 generated by the sensor 118. The plethysmograph waveform 164 may be displayed on the display 114 (shown in FIGS. 1 and 2).

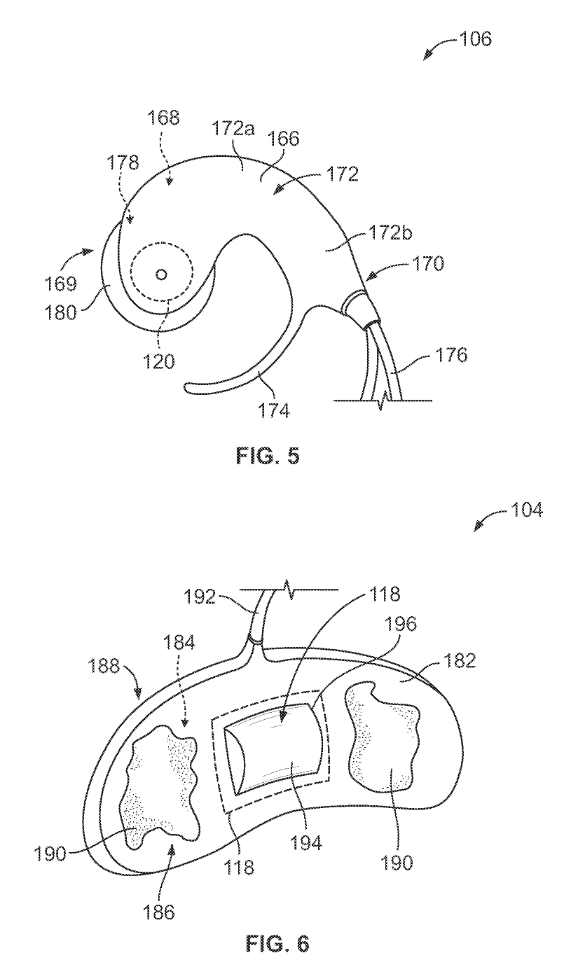

FIG. 5 is an elevational view of an exemplary embodiment of the temporal sensor device 106. The temporal sensor device 106 includes a housing 166 and the sensor 120. The sensor 120 is held by the housing 166. Specifically, the housing 166 includes an internal compartment 168 within which the sensor 120 is held. The housing 166 extends a length from an end 169 to an opposite end 170.

The length of the housing 166 defines an ear clip 172 that is configured to be received around the base of the patient's ear. Specifically, at least a portion of the length of the housing 166 extends along a path that is complementary with the shape of at least a portion of the base of the patient's ear. For example, as shown in FIG. 5, the ear clip 172 follows a curved path, which is complementary with the curved shape of the base of the patient's ear as the base extends from a front of the base to a back of the base. In the exemplary embodiment of FIG. 5, the end 169 of the housing 166 is configured to be positioned over the temporal artery of the patient in front of the base of the patient's ear. The ear clip 172 of the housing 166 extends outward from the end 169 along a path that is configured to wrap around a top of the base of the patient's ear. Specifically, the ear clip 172 includes a top segment 172a that extends from the end 169, which is configured to extend over the front of the base of the patient's ear. The top segment 172a is configured to extend over the top of the base of the patient's ear. The ear clip 172 includes a back segment 172b that extends from the top segment 172a and is configured to extend over a portion of the back of the base of the patient's ear. Although shown and described as an “end” of the housing 166, the end 169 may alternatively not be an “end” of the housing, but rather may be an intermediate segment of the housing 166 that extends (i.e., is connected) between two other segments of the housing 166. Accordingly, the end 169 may be referred to herein as a “temporal segment”.

The ear clip 172 may include a lower extension 174 that is configured to wrap around the back of the base of the patient's ear. In the exemplary embodiment of FIG. 5, the lower extension 174 extends outward from the back segment 172b and is configured to extend over a portion of the back and a portion of the bottom of the base of the patient' ear to facilitate providing a secure mechanical connection to the patient's ear. Accordingly, in the exemplary embodiment of FIG. 5, the lower extension 174 is configured to wrap around both the back and the bottom of the base of the patient's ear. The lower extension 174 may be integrally formed with the housing 166 (e.g., with the remainder of the ear clip 172), or the lower extension 172 may be a discrete component from the housing 166 that is mechanically connected (e.g., using a hinge and/or the like) to the housing 166. In some alternative embodiments, the lower extension 174 may not wrap around any portion of the bottom of the base of the patient's ear.

The various segments and the optional extension 174 of the ear clip 172 wrap around the base of the patient's ear to provide the temporal sensor device 106 with a secure fit to the patient's ear. In some embodiments, at least a portion of the housing 166 is resiliently deflectable such that the ear clip 172 is resiliently compressible around the base of the patient's ear. For example, the lower extension 174, the segment 172a, and/or the segment 172b may be formed as a spring to enable the ear clip 172 to grasp the base of the patient's ear by exerting a compression force on the ear base. The ear clip 172 may be provided in a variety of sizes and shapes to accommodate patient ears of different sizes and shapes. Each size and shape of ear clip 172 may accommodate a range of different ear sizes and/or shapes. Providing the ear clip 172 as resiliently compressible may facilitate accommodating a greater range of ear sizes and/or shapes.

The temporal sensor device 106 may include a cable 176 for communicating and/or drawing power from the workstation 102 and/or the modules 110 and/or 112. In addition or alternatively, the temporal sensor device 106 may communicate wirelessly with the workstation 102 and/or the modules 110 and/or 112. In addition or alternatively to drawing power from the workstation 102 and/or the modules 110 and/or 112, the temporal sensor device 106 may include a battery and/or any other suitable internal power source for providing power to various components thereof.

At least a portion of the sensor 120 is held within the internal compartment 168 of the housing 166 at the end 169 of the housing 166. Accordingly, the sensor 120 is positioned over the temporal artery of the patient in front of the base of the patient's ear. Such a position of the sensor 120 enables the sensor to detect plethysmograph waveforms from the temporal artery. The housing 166 may include a suitable window, transparent member, and/or other type of opening (not shown) that extends through a patient side 178 of the housing 166 to enable the sensor 120 to detect plethysmograph waveforms from the temporal artery from within the internal compartment 168.

The sensor 120 may draw power from the workstation 102 and/or the modules 110 and/or 112, for example via the optional cable 176 that operatively connects the temporal sensor device 106 to the workstation 102 and/or the modules 110 and/or 112. In addition or alternatively to drawing power from the workstation 102 and/or the modules 110 and/or 112, the temporal sensor device 106 may include a battery and/or any other suitable internal power source (not shown) for providing power to the sensor 120.

Although the exemplary embodiment of the sensor 120 is a PPG sensor, it should be understood that the configuration of the housing 166 and other components of the temporal sensor device 106 described and/or illustrated herein (e.g., with respect to FIG. 5) are applicable and suitable for use with other types of sensors, such as, but not limited to, blood pressure sensors, any other type of sensor that is configured to detect plethysmograph waveforms from an artery, and/or the like. For example, the temporal sensor device 106 shown in FIG. 5 and the various components thereof may be configured for use with a sensor that has any particular size; that has any particular shape; that is configured to detect plethysmograph waveforms in any manner; and/or the like.

Moreover, in embodiments wherein the sensor 120 is a PA sensor, the temporal sensor device 106 may include a coupling agent (not shown), for example held within the internal compartment 168 or another internal compartment. The coupling agent is configured to allow the transmission of both acoustic energy and light therethrough. The coupling agent may be any type of coupling agent that is configured to allow the transmission of both acoustic energy and light therethrough, such as, but not limited to, a gel media, a cream, a fluid, a paste, an ointment, an ultrasound gel, and/or the like. In some embodiments, the temporal sensor device 106 includes a sponge (not shown) or other matrix device that is impregnated with the coupling agent for holding the coupling agent. Exemplary coupling agents are described in U.S. patent application Ser. No. 13/612,160, filed on Sep. 12, 2012, entitled “PHOTOACOUSTIC SENSOR SYSTEM” (Attorney Docket No. H-RM-02755 (959-0531US1)), which is hereby incorporated by reference in its entirety.

The temporal sensor device 106 may include an adhesive 180 that extends on at least a portion of the patient side 178 of the housing 166. The adhesive 180 is configured to affix the end 169 of the housing 166 to the skin of the patient. The adhesive 180 thus further secures the temporal sensor device 106 in position over the temporal artery and on the patient's ear. Any type of adhesive 180 may be used. In some embodiments, the adhesive 180 is an adhesive that is specifically designed to adhere to human skin. Moreover, in addition or alternative to the adhesive 180, the housing 166 may be configured to be affixed to the patient's skin using any other suitable affixing structure, such as, but not limited to, using suction, using an intermediate bracket that is affixed to the patient's skin (using any suitable affixing structure) and is configured to hold the housing 166, and/or the like. In some alternative embodiments, no affixing structure is used besides the housing 166 itself (i.e., the ear clip 172 alone holds the sensor 120 in position over the temporal artery).

The housing 166 of the temporal sensor device 106 may be a single unitary body. But, the housing 166 may have any number of components. For example, in some embodiments, the housing 166 includes two or more shells that are connected together using any suitable type of mechanical connection, such as, but not limited to, using at least one of a hinge, a living hinge, a clam shell arrangement, a snap-fit connection, a press-fit connection, a slide tension (i.e., interference) connection, a threaded fastener, a latch, a lock, and/or the like. Fabricating the housing 166 using two or more shells may ease the positioning of the sensor 120 and/or other components within the internal compartment 168 of the housing 166. The housing 166 may be fabricated using any suitable method, process, and/or means, such as, but not limited to, using an overmold process such that the housing 166 is an over-molded housing, using a lamination process such that housing 166 includes two or more layers that are laminated together, and/or the like.

Optionally, the temporal sensor device 106 is disposable in that the temporal sensor device 106 is intended for a single use only. As used herein, the terms “disposable” and “single use” are intended to mean that a disposable, single use, temporal sensor device 106 is used for one and only one patient, and thereafter discarded. For example, a disposable, single use, temporal sensor device 106 may be used for one and only one measurement procedure on one and only one patient, and thereafter discarded. Alternatively, a disposable, single use, temporal sensor device 106 may be used for a plurality of measurement procedures on one and only one patient, and thereafter discarded. When used for a plurality of measurement procedures on one patient, the disposable, single use, temporal sensor device 106 is only applied to the patient one and only one time. However, the temporal sensor device 106 may be repositioned on the one and only one patient, for example, to accommodate different measurement locations for different measurements and/or to obtain more accurate measurements.

In other embodiments, all or a portion of the temporal sensor device 106 is re-usable with different patients. For example, the housing 166 and the sensor 120 may both be reusable together with different patients. Moreover, and for example, the housing 166 may be reusable with different patients while the sensor 120 may be replaced for each different patient or after use with a group of patients. Another example includes a reusable housing 166 and/or sensor 120 having disposable pads, strips, and/or the like of the adhesive 180 applied thereto for each use of the device 106.

The material(s), size, shape, thickness(es), and/or any other properties, attributes, and/or the like of the various components of the temporal sensor device 106 may be selected to facilitate providing and/or configuring the temporal sensor device 106 as disposable and single use.

FIG. 6 is a perspective view of an exemplary embodiment of the carotid sensor device 104. The carotid sensor device 104 includes a housing 182 and the sensor 118, which is held within an internal compartment 184 of the housing 182. The housing 182 includes a patient side 186 that is configured to face the patient's skin and an opposite side 188.

The housing 182 is configured to be positioned on a neck of the patient over a carotid artery of the patient. Specifically, the housing 182 is configured to be affixed to the patient's neck over the carotid artery using an adhesive 190. The adhesive 190 extends on at least a portion of the patient side 186 of the housing 182 for affixing the housing 182 to the skin of the patient. The adhesive 190 thus secures the carotid sensor device 104 in position over the carotid artery and on the patient's neck. Any type of adhesive 190 may be used. In some embodiments, the adhesive 190 is an adhesive that is specifically designed to adhere to human skin. In some alternative embodiments, the adhesive 190 is not used, and another type of fastener (e.g., a clip, a strap, a collar, using suction, using an intermediate bracket that is affixed to the patient's skin (using any suitable affixing structure) and is configured to hold the housing 182, and/or the like) holds the carotid sensor device 104 in position over the carotid artery.

In some embodiments, the patient side 186 of the housing 182 includes a surface having a curvature that is complementary with the curvature of the patient's neck. Moreover, in some embodiments, the housing 182 is at least partially flexible for complying to the shape of the patient's neck. Such a complementary curvature and/or flexible manner may facilitate a better fit between the carotid sensor device 104 and the patient's neck, which may enable the sensor 118 to more accurately detect plethysmograph waves from the carotid artery. The housing 182 may be provided in a variety of sizes and shapes to accommodate patient necks of different sizes and shapes. Each size and shape of the housing 182 may accommodate a range of different neck sizes and/or shapes.

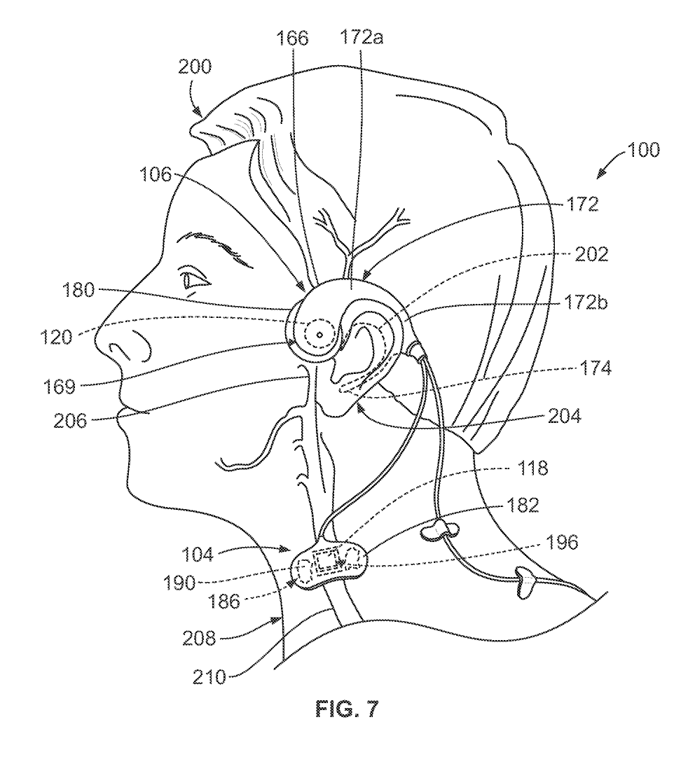

The carotid sensor device 104 may communicate and/or draw power from the workstation 102 and/or the modules 108 and/or 112, for example through the cable 176 (shown in FIGS. 5 and 7). In addition or alternatively, the carotid sensor device 104 may communicate wirelessly with the workstation 102 and/or the modules 108 and/or 112. In addition or alternatively to drawing power from the workstation 102 and/or the modules 108 and/or 112, the carotid sensor device 104 may include a battery and/or any other suitable internal power source (not shown) for providing power to various components thereof. In some embodiments, the carotid sensor device 104 is operatively connected directly to the workstation 102 and/or the modules 108 and/or 112 via a cable (not shown) that extends from the carotid sensor device 104 to the workstation 102 and/or the modules 108 and/or 112. In other words, in addition or alternatively to the cable 176, the sensor system 100 may include another cable that directly operatively connects the carotid sensor device 104 to the workstation 102 and/or the modules 108 and/or 112.

The carotid sensor device 104 is operatively connected to the temporal sensor device 106. For example, the carotid sensor device 104 may be operatively connected to the temporal sensor device 106 through a cable 192. In addition or alternatively, the carotid sensor device 104 may communicate with the temporal sensor device 106 wirelessly. In other embodiments, the carotid sensor device 104 and the temporal sensor device 106 may not communicate directly with each other, and/or may be operatively connected through the workstation 102 and/or the modules 108, 110, and/or 112.

At least a portion of the sensor 118 is held within the internal compartment 184 of the housing 182 such that the sensor 118 is positioned over the carotid artery of the patient on the patient's neck. Such a position of the sensor 118 enables the sensor to detect plethysmograph waveforms from the carotid artery. The housing 182 includes a transparent member 194 that provides a window on the patient side 186 of the housing 182 that enables the sensor 118 to detect plethysmograph waveforms from the temporal artery from within the internal compartment 184. In addition or alternatively to the transparent member 194, the housing 182 may include any other suitable window, transparent member, and/or other type of opening (not shown) that enables the sensor 118 to detect plethysmograph waveforms from the carotid artery from within the internal compartment 184.

The patient side 186 of the housing 182 may include a convex segment 196 that engages the patient's skin. The convex segment 196 is located along the patient side 186 at the window. The convex segment 196 is configured to engage the patient's skin over the carotid artery such that the convex segment 196 locates the sensor 118 relative to the carotid artery for detecting plethysmograph waveforms therefrom.

The sensor 118 may draw power from the workstation 102, the modules 110 and/or 112, and/or the temporal sensor device 106, for example via the optional cable 176 and/or the optional cable 192, respectively. In addition or alternatively to drawing power from the workstation 102, the modules 110 and/or 112, and/or the temporal sensor device 106, the carotid sensor device 104 may include a battery and/or any other suitable internal power source (not shown) for providing power to the sensor 118.

Although the exemplary embodiment of the sensor 118 is a blood pressure sensor, it should be understood that the configuration of the housing 182 and other components of the carotid sensor device 104 described and/or illustrated herein (e.g., with respect to FIG. 6) are applicable and suitable for use with other types of sensors, such as, but not limited to, PPG sensors, any other type of sensor that is configured to detect plethysmograph waveforms from an artery, and/or the like. For example, the carotid sensor device 104 shown in FIG. 6 and the various components thereof may be configured for use with a sensor that has any particular size; that has any particular shape; that is configured to detect plethysmograph waveforms in any manner; and/or the like. Moreover, each of the housing 182 and the window (i.e., the transparent member 194) may have any other shape than is shown herein.

Moreover, in embodiments wherein the sensor 118 is a PA sensor, the carotid sensor device 104 may include a coupling agent (not shown), for example held within the internal compartment 184 or another internal compartment. The coupling agent is configured to allow the transmission of both acoustic energy and light therethrough. The coupling agent may be any type of coupling agent that is configured to allow the transmission of both acoustic energy and light therethrough, such as, but not limited to, a gel media, a cream, a fluid, a paste, an ointment, an ultrasound gel, and/or the like. In some embodiments, the carotid sensor device 104 includes a sponge (not shown) or other matrix device that is impregnated with the coupling agent for holding the coupling agent.

The housing 182 of the carotid sensor device 104 may be a single unitary body. But, the housing 182 may have any number of components. For example, in some embodiments, the housing 182 includes two or more shells that are connected together using any suitable type of mechanical connection, such as, but not limited to, using at least one of a hinge, a living hinge, a clam shell arrangement, a snap-fit connection, a press-fit connection, a slide tension (i.e., interference) connection, a threaded fastener, a latch, a lock, and/or the like. Fabricating the housing 182 using two or more shells may ease the positioning of the sensor 118 and/or other components within the internal compartment 184 of the housing 182. Moreover, in other embodiments, the housing 182 includes two or more layers of fabric, plastic, adhesive, plastic adhesive, and/or other materials that are laminated together with the sensor 118. The housing 182 may be fabricated using any suitable method, process, and/or means, such as, but not limited to, using an overmold process such that the housing 182 is an over-molded housing, using a lamination process such that housing 182 includes two or more layers that are laminated together, and/or the like.

Optionally, the carotid sensor device 104 is disposable in that the carotid sensor device 104 is intended for a single use only. In other embodiments, all or a portion of the carotid sensor device 104 is re-usable with different patients. For example, the housing 182 and the sensor 118 may both be reusable together with different patients. Moreover, and for example, the housing 182 may be reusable with different patients while the sensor 118 may be replaced for each different patient or after use with a group of patients. Another example includes a reusable housing 182 and/or sensor 118 having disposable pads, strips, and/or the like of the adhesive 190 applied thereto for each use of the device 104. The material(s), size, shape, thickness(es), and/or any other properties, attributes, and/or the like of the various components of the carotid sensor device 104 may be selected to facilitate providing and/or configuring the carotid sensor device 104 as disposable and single use.

FIG. 7 is an elevational view illustrating the sensor system 100 operatively connected to a patient 200. The ear clip 172 of the temporal sensor device 106 is wrapped around a base 202 of an ear 204 of the patient 200. Specifically, the end 169 of the housing 166 extends in front of the base 202 of the patient's ear 204. The top segment 172a of the ear clip 172 extends over the top of the base 202 of the patient's ear 204, while the back segment 172b extends over a portion of the back of the base 202. The lower extension 174 is wrapped around a portion of the back and a portion of the bottom of the base 202 of the patient's ear 204. The ear clip 172 thus provides the temporal sensor device 106 with a secure fit to the patient's ear 204. The end 169 of the housing 166 is positioned over a temporal artery 206 of the patient 200 such that the sensor 120 is positioned to detect plethysmograph waveforms from the temporal artery 206. As described above, the end 169 of the housing 166 may be affixed to the patient's skin using the adhesive 180. Although shown as being attached to a left ear 204 of the patient 200 the temporal sensor device 106 may alternatively be configured to be attached to a right ear (not shown) of the patient 200, or may be configured for selective attachment to both the right ear and the left ear 204.

The carotid sensor device 104 is affixed to a neck 208 of the patient 200 neck over a carotid artery 210 of the patient 200. Specifically, the patient side 186 of the housing 182 of the carotid sensor device 104 is affixed to the patient's skin using the adhesive 190. The convex segment 196 of the patient side 186 of the housing 182 is engaged with the patient's skin over the carotid artery 210 to locate the sensor 118 relative to the carotid artery 210. Specifically, when the convex segment 196 is engaged with the patient's skin over the carotid artery 210, the sensor 118 is positioned over the carotid artery 210 such that the sensor 118 is configured to detect plethysmograph waveforms from the carotid artery 210. The carotid sensor device 104 may be attached to either side of the patient's neck 208.

As can be seen in FIG. 7, both the carotid sensor device 104 and the temporal sensor device 106 are affixed to the patient 200 externally for detecting plethysmograph waveforms through the patient's skin. Accordingly, each of the sensor devices 104 and 106 provides a non-invasive sensor that is configured to detect plethysmograph waveforms from an artery in a non-invasive manner.

The plethysmograph waveforms detected by the devices 104 and 106 may be used by the workstation 102 and/or the modules 108, 110, and/or 112 to determine a pulse transit time measurement of the patient 200. For example, the workstation 102 and/or the modules 108, 110, and/or 112 may compare one or more plethysmograph waveforms from the carotid sensor device 104 with one or more plethysmograph waveforms from the temporal sensor device 106 to determine one or more pulse transit time measurements.

One example of determining a pulse transit time measurement includes using a time delay between a plethysmograph waveform from the carotid sensor device 104 and a plethysmograph waveform from the temporal sensor device 106. For example, because arterial wall stiffness increases with pressure, the pulse-wave velocity traveling down the radial artery increases with increasing pulse pressure. Pulse pressure is a function of stroke volume and peripheral vascular resistance. For example, pulse wave velocity, pulse pressure, and cardiac output may be given by the following equations (1), (2), and (3), respectively:

PWV=a·(PP+b) (1)

PP=SV·PVR (2)

CO=SV−HR (3)

where PWV is pulse wave velocity, PP is pulse pressure, SV is stroke volume, PVR is peripheral vascular resistance, CO is cardiac output, HR is heart rate, and a, b, and c are empirically determined constants. The time delay between the plethysmograph waveform from the carotid sensor device 104 and the plethysmograph waveform from the temporal sensor device 106 can be used to determine a pulse transit time measurement, for example in accordance with the following equation:

PWV=x/TD (4)

where x is the effective vascular distance between the sensors 118 and 120, and TD is the time delay, which may be a pulse transit time. The effective vascular distance x may be determined from a look-up table of the patient's height, weight, and age based on empirically-derived anatomical statistics.

The sensor system 100 is not limited to the exemplary methods, algorithms, and/or the like described herein for determining pulse transit time measurements using the plethysmograph waveforms from the sensor devices 104 and 106. Rather, any other methods, algorithms, and/or the like may be used to determine pulse transit time measurements using the plethysmograph waveforms from the sensor devices 104 and 106. Because the plethysmograph waveforms are detected at relatively close locations within the vasculature of the patient 200 (i.e., from the carotid and temporal arteries 210 and 206, respectively) and/or because the path between the locations is relatively uncomplicated, the sensor system 100 may provide a more accurate determination of pulse transit time.

The pulse transit time measurement of the patient 200 may be used to determine various physiological parameters of the patient, such as, but not limited to, pulse pressure, cardiac output, stroke volume, vascular resistance, and/or the like. For example, the pulse time transit measurement may be used by the workstation 102 and/or the modules 108, 110, and/or 112 to determine both a pulse pressure (i.e., a driving pressure) of the patient 200 and peripheral vascular resistance. The pulse pressure and peripheral vascular resistance can then be used to determine various physiological parameters of the patient 200, such as, but not limited to, cardiac output, stroke volume, and/or the like.

One example of using pulse pressure and peripheral vascular resistance includes determining pulse pressure using the following equation:

$$PP = \frac{x}{a * T D} - b \qquad ( 5 )$$

where a and b are the empirically derived constants of equation (1). The peripheral vascular resistance can be determined by an ensemble averaged pressure pulse derived from the plethysmograph waveform of the carotid sensor device 104 and/or derived from the plethysmograph waveform of the temporal sensor device 106. The ensemble pressure pulse, P(t), can be approximated by two curves, P₁(t) and P₂(t), through curve fitting, such that:

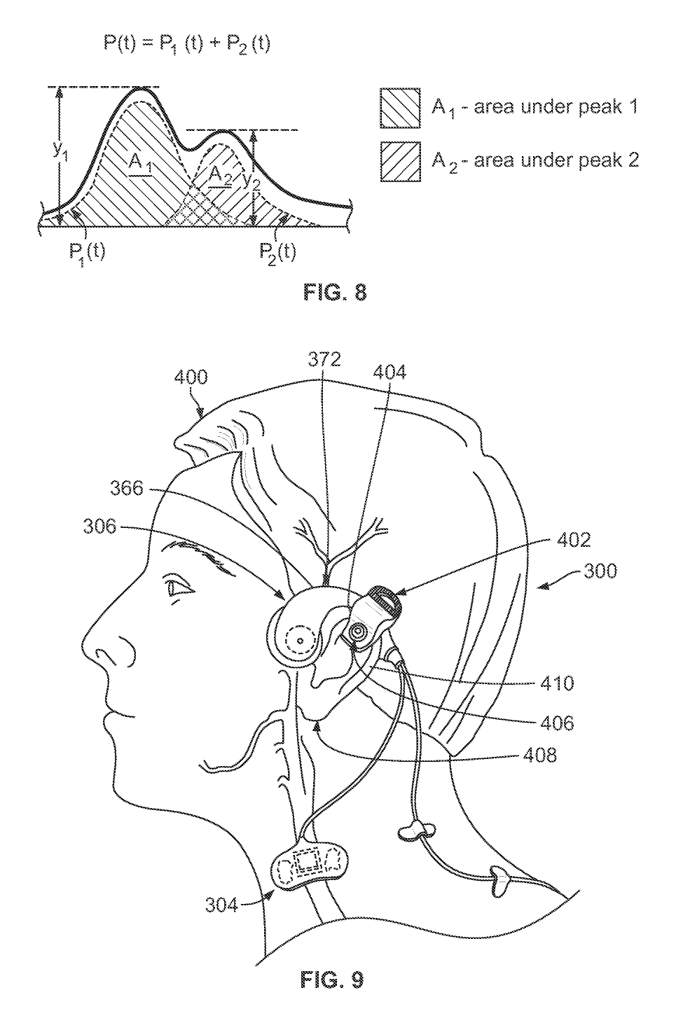

P(t)=P₁(t)+P₂(t) (6)

where P₁(t) is a first curve with peak amplitude y₁ and P₂(t) is a second curve with peak amplitude y₂ as shown in FIG. 8. The heights of the two peaks and the areas under the two peaks are the morphology features used to determine stroke volume and peripheral vascular resistance. For example, the relative size of the reflected wave and primary wave is determined by the peripheral vascular resistance, which can be approximated by the equation:

$$P V R = {C ( \frac{{A}_{2}}{{A}_{1}} )}^{∝} \qquad ( 7 )$$

where PVR is the peripheral vascular resistance, C and α are empirically determined parameters, A₁ is the area under the first peak, and A₂ is the area under the second peak.

The stroke volume can then be calculated, for example, by substituting the peripheral vascular resistance and pulse pressure into equation (2), which gives the following equation:

$$SV = \frac{\frac{x}{a * T D} - b}{{C ( \frac{{A}_{2}}{{A}_{1}} )}^{∝}} \qquad ( 8 )$$

Because heart rate is a parameter that may be relatively easily calculated from the plethysmograph waveforms of the devices 104 and/or 106, cardiac output may be given by equation (3).

The sensor system 100 is not limited to the exemplary methods, algorithms, and/or the like described herein for determining various physiological parameters of the patient 200 using pulse transit time measurements. Rather, any other methods, algorithms, and/or the like may be used to determine various physiological parameters of the patient 200 using pulse transit time measurements.

Although shown as being located over the carotid and temporal arteries on the patient's neck and in front of the patient's ear, respectively, the sensor system 100 is not limited to such locations. Rather, the sensor devices 104 and 106 (and any other sensor devices) of the sensor system 100 may have other locations along the patient's vasculature, such as, but not limited to, on a patient's wrist, on a patient's digit (e.g., a finger, a toe, a thumb, and/or the like), over a patient's ankle, and/or the like.

FIG. 9 is an elevational view of another embodiment of a sensor system 300 mounted on a patient 400. The sensor system 300 includes a temporal sensor device 306 and a carotid sensor device 304. The devices 304 and 306 are substantially similar to the devices 104 and 106 shown in FIGS. 5 and 6, respectively. In addition to the devices 304 and 306, the sensor system 300 includes a pulse oximeter sensor device 402.

A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Pulse oximeters may also be used to measure the pulse rate of a patient. Pulse oximeters typically measure and display various blood flow characteristics including, but not limited to, the oxygen saturation of hemoglobin in arterial blood.

In the exemplary embodiment of FIG. 9, the pulse oximeter sensor device 402 is held by the temporal sensor device 306. Specifically, the pulse oximeter sensor device 402 includes a clip 404 that extends from a housing 366 of the temporal sensor device 306. The pulse oximeter sensor device 402 includes a pulse oximeter sensor 406 that is held by the clip 404. When an ear clip 372 of the temporal sensor device 306 is affixed to an ear 408 of the patient 400, the pulse oximeter sensor 406 is held by the clip 404 such that the pulse oximeter sensor 406 is positioned on a lobe 410 of the patient's ear 408 for detecting pulse oximeter waveforms. The clip 404 may be integrally formed with the housing 366 of the temporal sensor device 306, or the clip 404 may be a discrete component from the housing 366 that is mechanically connected (e.g., using a hinge and/or the like) to the housing 366. In addition or alternatively to the clip 404, the pulse oximeter sensor device 402 may include any other structure, means, and/or the like for holding the pulse oximeter sensor 406.

The pulse oximeter sensor 406 may include a light sensor (not shown; e.g., an emitter and a detector) that is placed at a site on the patient 400. The pulse oximeter sensor may pass light using a light source (not shown) through blood perfused tissue and photoelectrically sense the absorption of light in the tissue and/or blood. A signal representing light intensity versus time or a mathematical manipulation of this signal (for example, a scaled version thereof, a log taken thereof, a scaled version of a log taken thereof, and/or the like) may be referred to as the pulse oximeter waveform. In addition, the term “pulse oximeter waveform,” as used herein, may also refer to an absorption signal (for example, representing the amount of light absorbed by the tissue and/or blood) or any suitable mathematical manipulation thereof.

The pulse oximeter waveforms detected by the pulse oximeter sensor device 402 may be used in combination with the plethysmograph waveforms from the carotid sensor device 304 and/or with the plethysmograph waveforms from the temporal sensor device 306 for determining a pulse transit time measurement of the patient 400. The pulse oximeter waveforms of the sensor device 402 may provide a third source of information for determining pulse transit time measurements. Moreover, the pulse oximeter waveform may provide a different type of waveform for comparison to the plethysmograph waveforms of the devices 304 and 306. The greater amount of information provided by the three sensors (i.e., the sensing devices 304, 306, and 402) and/or the different type of waveform provided by the pulse oximeter sensor device 402 may enable embodiments of the present disclosure to be more accurate and/or reliable than previous systems.

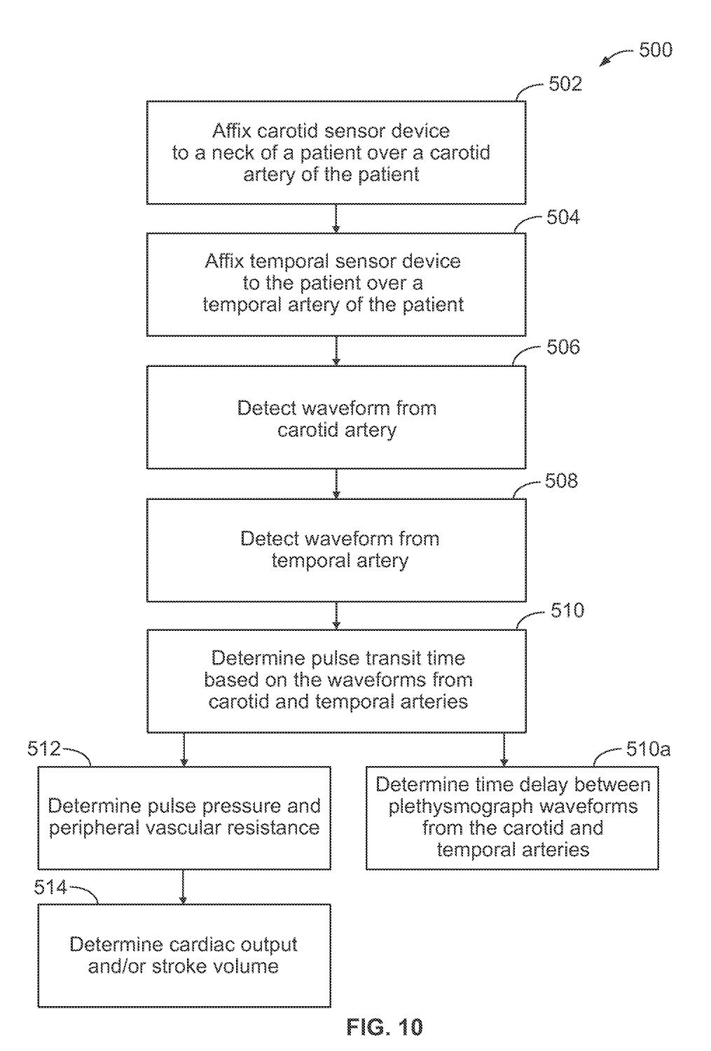

FIG. 10 is a flowchart illustrating an exemplary embodiment of a method 500 for determining a pulse transit time measurement of a patient (e.g., the patient 200 shown in FIG. 7 or the patient 400 shown in FIG. 9) using a sensor system (e.g., the sensor system 100 shown in FIGS. 1, 2, and 7 or the sensor system 300 shown in FIG. 9). The method 500 includes, at 502, affixing a carotid sensor device (e.g., the carotid sensor device 104 shown in FIGS. 1, 2, 6, and 7 or the carotid sensor device 304 shown in FIG. 9) to a neck (e.g., the neck 208 shown in FIG. 7) of the patient over a carotid artery (e.g., the carotid artery 210 shown in FIG. 7) of the patient. In some embodiments, affixing the carotid sensor device to the neck of the patient at 502 includes the carotid sensor device to the neck using an adhesive.

At 504, the method includes affixing a temporal sensor device (e.g., the temporal sensor device 106 shown in FIGS. 1, 2, 5, and 7 or the temporal sensor device 306 shown in FIG. 9) to the patient over a temporal artery (e.g., the temporal artery 206 shown in FIG. 7) of the patient. In some embodiments, affixing the temporal sensor device to the patient at 504 comprises receiving an ear clip of the temporal sensor device over an ear of the patient.

At 506, the method 500 includes detecting a plethysmograph waveform from the carotid artery of the patient using the carotid sensor device. The method 500 also includes detecting, at 508, a plethysmograph waveform from the temporal artery of the patient using the temporal sensor device.

At 510, the method 500 includes determining the pulse transit time measurement based, at least in part, on the plethysmograph waveforms from the carotid and temporal arteries. Determining at 510 the pulse transit time measurement may include determining, at 510a, a time delay between the plethysmograph waveform from the carotid artery the plethysmograph waveform from the temporal artery. For example, in some embodiments, determining at 510 the pulse transit time includes dividing a vascular distance between the carotid and temporal sensor devices by a time delay between the plethysmograph waveform from the carotid artery the plethysmograph waveform from the temporal artery.

The method 500 may include, at 512, determining a pulse pressure and a peripheral vascular resistance, at least in part, from the pulse transit time measurement. At 514, the method 500 may include determining a cardiac output and/or a stroke volume using the pulse pressure and the peripheral vascular resistance.

Certain embodiments of the present disclosure may provide a sensor system that is more accurate and reliable than previous systems for determining pulse transit time measurements, cardiac output, stroke volume, vascular resistance, and/or the like. Embodiments of the present disclosure may provide a sensor system for determining pulse transit time measurements that includes at least two sensors that are spaced apart along the vasculature of the patient. The greater amount of information provided by the at least two sensors may enable embodiments of the present disclosure to be more accurate and/or reliable than previous systems that determined pulse transit time measurements using a single sensor location. Certain embodiments of the present disclosure may provide a sensor system that detects plethysmograph waveforms at relatively close locations having a path therebetween that is relatively direct and uncomplicated. The relatively direct and uncomplicated path (e.g., from the carotid artery to the temporal artery, or vice versa) may result in less propagation errors in the determined pulse transit time than longer, indirect, and/or more tortuous paths, for example measurement locations on a digit of the patient. The relatively direct and uncomplicated path may enable embodiments of the present disclosure to be more accurate and/or reliable than previous systems that utilizing longer, indirect, and/or more tortuous paths. Certain embodiments of the present disclosure may provide a sensor system that is less susceptible to vasoconstriction, for example because the measurement locations are taken along relatively large diameter segments (e.g., the temporal and carotid arteries) of the patient and not from the periphery (e.g., a finger or toe) where the effects of changing vasotone are most pronounced.

Certain embodiments of the present disclosure may provide a sensor system that enables sensors to detect plethysmograph waveforms from carotid and temporal arteries of a patient in a relatively non-invasive manner. Detection of the plethysmograph waveforms from the carotid and temporal arteries of the patient may be less invasive than at least some known sensor systems, and may cause the patient less discomfort, injury, and/or inconvenience.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, front, back, lower, mid, lateral, horizontal, vertical, and/or the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

What is claimed is:

1. A sensor system for determining a pulse transit time measurement of a patient, the sensor system comprising:
a carotid sensor device configured to be positioned on a neck of the patient over a carotid artery of the patient, the carotid sensor device being configured to detect a plethysmograph waveform from the carotid artery; and
a temporal sensor device operatively connected to the carotid sensor device, the temporal sensor device being configured to be positioned on the patient over a temporal artery of the patient, wherein the temporal sensor device is configured to detect a plethysmograph waveform from the temporal artery.

2. The sensor system of claim 1, further comprising a pulse transit time determination module operatively connected to the carotid sensor device and the temporal sensor device, the pulse transit time determination module being configured to determine the pulse transit time measurement based, at least in part, on the plethysmograph waveforms from the carotid and temporal arteries.

3. The sensor system of claim 1, wherein the temporal sensor device comprises a housing and a sensor held by the housing, the sensor being configured to detect the plethysmograph waveform from the temporal artery, the housing defining an ear clip that is configured to be received around the base of an ear of the patient.

4. The sensor system of claim 1, wherein the temporal sensor device comprises a housing and a sensor held by the housing, the sensor being configured to detect the plethysmograph waveform from the temporal artery, the housing comprising an ear clip having an end that is configured to be positioned over the temporal artery of the patient, the ear clip extending outward from the end along a path that is configured to wrap around a top of a base of an ear of the patient.

5. The sensor system of claim 1, wherein the temporal sensor device comprises a housing and a sensor held by the housing, the sensor being configured to detect the plethysmograph waveform from the temporal artery, the housing defining an ear clip that is configured to be received around the base of an ear of the patient, the ear clip comprising a lower extension that is configured to wrap around a back of the base of the patient's ear.

6. The sensor system of claim 1, wherein the temporal sensor device comprises a housing and a sensor held by the housing, the sensor being configured to detect the plethysmograph waveform from the temporal artery, the housing defining an ear clip that is configured to be received around the base of an ear of the patient, the ear clip being resiliently compressible around the base of the patient's ear.

7. The sensor system of claim 1, wherein the carotid sensor device comprises a housing and a sensor held by the housing, the sensor being configured to detect the plethysmograph waveform from the carotid artery, the housing comprising a surface that includes a shape that is complementary with a shape of the patient's neck.

8. The sensor system of claim 1, wherein the carotid sensor device comprises a housing and a sensor held by the housing, the sensor being configured to detect the plethysmograph waveform from the carotid artery, the housing comprising a surface that includes a convex segment that is configured to engage skin of the patient's neck over the carotid artery.

9. The sensor system of claim 1, further comprising a cable, the carotid sensor device being operatively connected to the temporal sensor device via the cable.

10. The sensor system of claim 1, further comprising a pulse-oximeter sensor device that is held by the temporal sensor device such that the pulse-oximeter sensor device is configured to be positioned on a lobe of an ear of the patient for detecting pulse oximeter waveforms.

11. The sensor system of claim 1, wherein at least one of the carotid sensor device or the temporal sensor device comprises an adhesive for affixing the device to skin of the patient.

12. The sensor system of claim 1, wherein the carotid sensor device comprises at least one of a photoplethysmograph (PPG) sensor, a blood pressure sensor, a pressure transducer, an optical PPG sensor, a photoacoustic sensor, or a photon density wave sensor.

13. The sensor system of claim 1, wherein the temporal sensor device comprises at least one of a photoplethysmograph (PPG) sensor, a blood pressure sensor, a pressure transducer, an optical PPG sensor, a photoacoustic sensor, or a photon density wave sensor.

14. The sensor system of claim 1, wherein at least one of the carotid sensor device or the temporal sensor device is at least one of a non-invasive sensor device or a disposable, single use, sensor device.

15. A method for determining a pulse transit time of a patient using a sensor system, the method comprising:
affixing a carotid sensor device to a neck of the patient over a carotid artery of the patient;
affixing a temporal sensor device to the patient over a temporal artery of the patient;
detecting a plethysmograph waveform from the carotid artery of the patient using the carotid sensor device;
detecting a plethysmograph waveform from the temporal artery of the patient using the temporal sensor device; and
determining the pulse transit time measurement based, at least in part, on the plethysmograph waveforms from the carotid and temporal arteries.

16. The method of claim 15, wherein determining the pulse transit time measurement based, at least in part, on the plethysmograph waveforms from the carotid and temporal arteries comprises determining a time delay between the plethysmograph waveform from the carotid artery the plethysmograph waveform from the temporal artery.

17. The method of claim 15, wherein determining the pulse transit time measurement based, at least in part, on the plethysmograph waveforms from the carotid and temporal arteries comprises dividing a vascular distance between the carotid and temporal sensor devices by a time delay between the plethysmograph waveform from the carotid artery the plethysmograph waveform from the temporal artery to determine a pulse wave velocity.

18. The method of claim 15, further comprising:
determining a pulse pressure and a peripheral vascular resistance, at least in part, from the pulse transit time measurement; and
determining at least one of a cardiac output or a stroke volume using the pulse pressure and the peripheral vascular resistance.

19. The method of claim 15, wherein:
affixing the carotid sensor device to the neck of the patient over the carotid artery of the patient comprises attaching the carotid sensor device to the neck using an adhesive; and
affixing the temporal sensor device to the patient over the temporal artery of the patient comprises receiving an ear clip of the temporal sensor device over an ear of the patient.

20. A temporal sensor device comprising:
a housing comprising an internal compartment and a temporal segment, the housing comprising an ear clip that is configured to wrap around the base of an ear of a patient such that the temporal segment of the housing is positioned over a temporal artery of the patient; and
a sensor held within the internal compartment of the housing at the temporal segment of the housing such that the sensor is configured to detect a plethysmograph waveform from the temporal artery when the ear clip is wrapped around the base of the patient's ear.
